(19) United States
(12) Patent Application Publication
(10) Pub. No.: US 2025/0295816 A1
(43) Pub. Date: Sep. 25, 2025

(54) RADIOPAQUE AMINO-FUNCTIONAL CROSSLINKING AGENTS FOR MEDICAL APPLICATIONS

(71) Applicant: Boston Scientific Scimed, Inc., Maple Grove, MN (US)

(72) Inventors: Yen-Hao Hsu, Shrewsbury, MA (US); Heather Cirka, Hudson, MA (US); Nicolas Ball-Jones, Minneapolis, MN (US); Rick Dorn, Plymouth, MN (US); Joseph Thomas Delaney, JR., Minneapolis, MN (US); Cristian Parisi, Amherst, MA (US)

(73) Assignee: Boston Scientific Scimed, Inc., Maple Grove, MN (US)

(21) Appl. No.: 19/084,378

(22) Filed: Mar. 19, 2025

Related U.S. Application Data
(60) Provisional application No. 63/567,701, filed on Mar. 20, 2024.
(60) Provisional application No. 63/717,411, filed on Nov. 7, 2024.

(51) Int. Cl. A61K 49/04

(52) U.S. Cl. CPC A61K 49/0438; A61K 49/0457

(57) ABSTRACT

In some aspects, the present disclosure pertains to systems for forming hydrogels that comprise (a) a radiopaque polyamino compound and (b) a reactive polymer comprising a plurality of hydrophilic polymer segments and a plurality of reactive moieties, wherein the reactive moieties are reactive with amino groups of the radiopaque polyamino compound, and wherein the radiopaque polyamino compound is produced by a method that comprises: (i) converting hydroxyl groups of a polyhydroxylated radiopaque compound to amino groups, (ii) converting vicinal diol groups (—CHOHCH₂OH groups) of a polyhydroxylated radiopaque compound into hydroxyaminoethyl groups (—CHOHCH₂NH₂ groups), or (iii) converting vicinal diol groups of a polyhydroxylated radiopaque compound into aminomethyl groups (—CH₂NH₂ groups). In other aspects, the present disclosure pertains to hydrogels formed from such systems and to methods of treatment using such systems.

### CROSS-REFERENCE TO RELATED APPLICATIONS

The application claims the benefit of U.S. Provisional Patent Application Ser. No. 63/717,411 filed on Nov. 7, 2024, and U.S. Provisional Patent Application Ser. No. 63/567,701 filed on Mar. 20, 2024, the disclosures of which are incorporated herein by reference.

### FIELD

The present disclosure relates to radiopaque amino-functional crosslinking agents, to methods of forming radiopaque amino-functional crosslinking agents, and to medical hydrogels formed from such radiopaque amino-functional crosslinking agents.

### BACKGROUND

SpaceOAR®, a rapid crosslinking hydrogel that polymerizes in vivo within seconds, is based on a multi-arm polyethylene glycol (PEG) polymer with a polyol core functionalized with succinimidyl glutarate as reactive end groups which further react with trilysine to form crosslinks. This product has become a very successful, clinically used biomaterial in prostate cancer therapy. A further improvement based on this structure is that a portion of the succinimidyl glutarate end groups have been replaced with 2,3,5-triiodobenzamide groups, providing radiopacity. This hydrogel, known by the trade name of SpaceOAR Vue®, is the radiopaque version of SpaceOAR® for prostate medical applications. Above a specific pH, the succinimidyl glutarate groups of SpaceOAR® and SpaceOAR Vue® will rapidly react with the trilysine crosslinker in vivo to form a hydrogel. The hydrogel breaks down in-vivo over the course of ca. 6-9 months. The breakdown occurs primarily through the hydrolysis of the ester linkages on the glutarate groups.

The present disclosure provides an alternative to the trilysine crosslinker of SpaceOAR®, which is costly and difficult to prepare. The present disclosure also provides an alternative to replacing a portion of the succinimidyl glutarate end groups with 2,3,5-triiodobenzamide groups when radiopacity is desired.

### SUMMARY

The above and other benefits are provided in the present disclosure by replacing the trilysine crosslinker with an alternative polyamino crosslinker that provides the resulting hydrogel with the desired radiopacity.

In some aspects, the present disclosure pertains to systems for forming hydrogels that comprise (a) a radiopaque polyamino compound and (b) a reactive polymer comprising a plurality of hydrophilic polymer segments and a plurality of reactive moieties, wherein the reactive moieties are reactive with amino groups of the radiopaque polyamino compound, and wherein the radiopaque polyamino compound is produced by a method that comprises: (i) converting hydroxyl groups (—OH groups) of a polyhydroxylated radiopaque compound to amino groups (—NH₂ groups), (ii) converting vicinal diol groups (—CHOHCH₂OH groups) of a polyhydroxylated radiopaque compound into hydroxyaminoethyl groups (—CHOHCH₂NH₂ groups), or (iii) converting vicinal diol groups of a polyhydroxylated radiopaque compound into aminomethyl groups (—CH₂NH₂ groups).

In some embodiments, the polyhydroxylated radiopaque compound comprises at least one hydroxy-substituted iodinated aromatic group. In some of these embodiments, the at least one hydroxy-substituted iodinated aromatic group comprises a monocyclic or multicyclic aromatic group that is substituted with a plurality of iodine groups and a plurality of hydroxyalkyl-containing groups. The plurality of hydroxyalkyl-containing groups may comprise, for example, C₂-C₆-hydroxyalkyl groups, which in some embodiments may comprise at least one vicinal diol group. In some embodiments, the plurality of hydroxyalkyl-containing groups may comprise, for example, at least one hydroxyalkyl-containing group that is linked to the monocyclic or multicyclic aromatic group through an amide-containing linkage.

In some embodiments, which can be used in conjunction with any of the above aspects and embodiments, the polyhydroxylated radiopaque compound may be selected for example, from

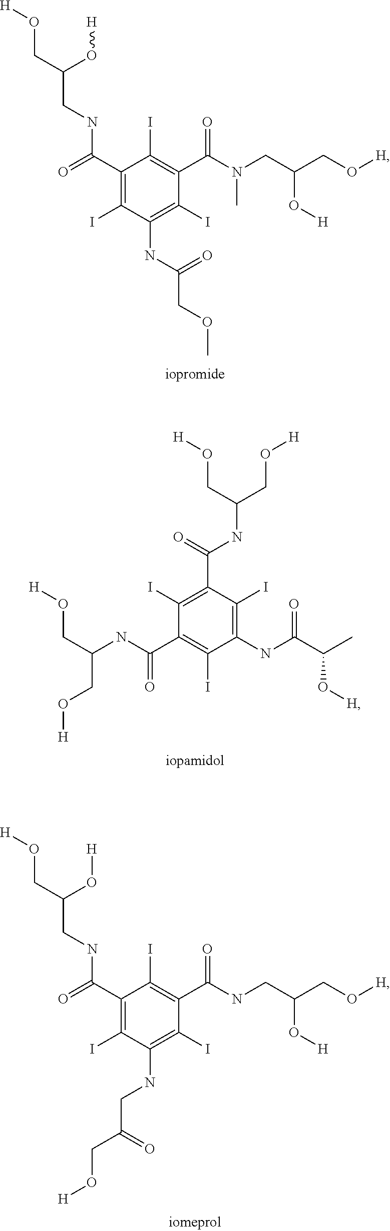

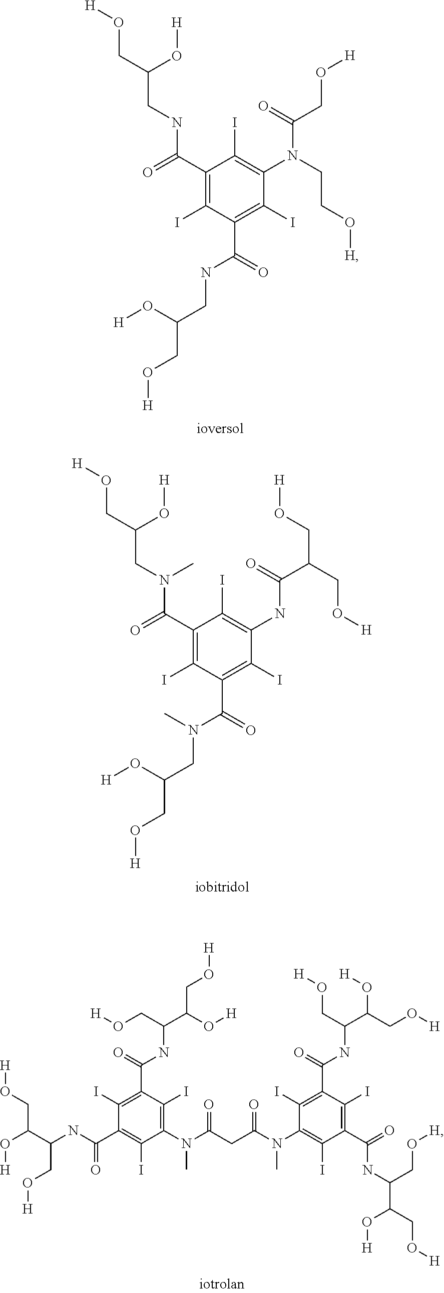

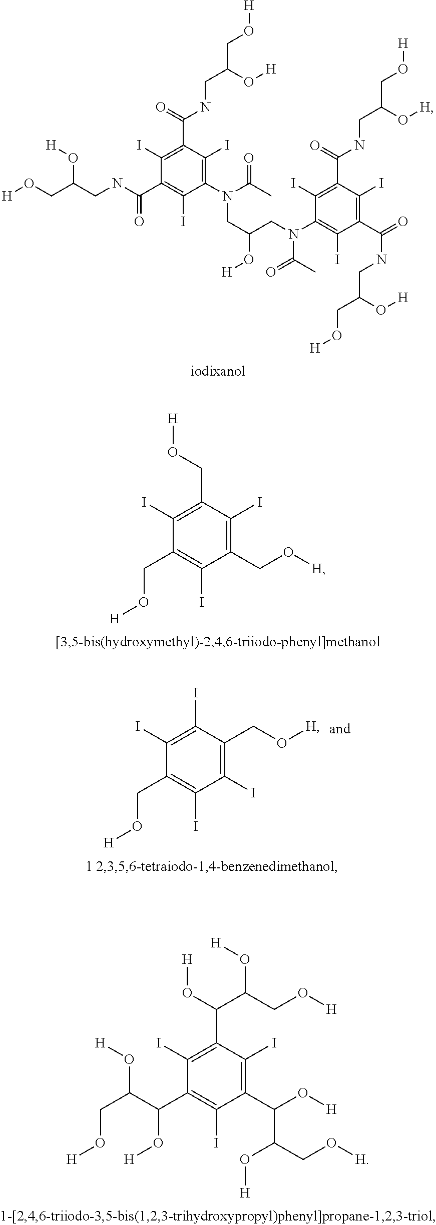

In some embodiments, which can be used in conjunction with any of the above aspects and embodiments, the radiopaque polyamino compound comprises at least one amino-substituted iodinated aromatic group. In some of these embodiments, the at least one amino-substituted iodinated aromatic group comprises a monocyclic or multicyclic aromatic group that is substituted with a plurality of iodine groups and plurality of amino-containing groups. In some embodiments, the plurality of amino-containing groups comprises aminoalkyl groups that comprise one or more amino groups and one or more carbon atoms. The aminoalkyl groups may comprise, for example, at least one C₂-C₆-aminoalkyl group that comprises two amino groups positioned on adjacent carbon atoms or at least one C₂-C₆-aminoalkyl group that comprises a single terminal amino group. In some embodiments, the plurality of amino-containing groups comprises hydroxyaminoalkyl groups that comprise one or more amino groups, one or more hydroxyl groups and one or more carbon atoms. In some of these embodiments, the hydroxyaminoalkyl groups comprise at least one C₂-C₆-hydroxyaminoalkyl group that comprises a hydroxyl group and an amino group positioned on adjacent carbon atoms. In some embodiments, the plurality of amino-containing groups comprises at least one amino-containing group that is linked to the monocyclic or multicyclic aromatic group through an amide-containing linkage.

In some embodiments, which can be used in conjunction with any of the above aspects and embodiments, the reactive moieties comprise an electrophilic moiety.

In some embodiments, which can be used in conjunction with any of the above aspects and embodiments, the reactive polymer is a multi-arm polymer that comprises three or more polymer arms linked to a core region, each arm comprising one of the hydrophilic polymer segments and one of the reactive moieties that are reactive with the amino groups of the radiopaque polyamino compound to form covalent crosslinks.

In some embodiments, which can be used in conjunction with any of the above aspects and embodiments, the reactive polymer is a multi-arm polymer that comprises three or more polymer arms linked to a core region, each arm comprising a cyclic anhydride residue disposed between one of the hydrophilic polymer segments and one of the reactive moieties, wherein the reactive moieties comprise a cyclic imide ester group.

In some embodiments, which can be used in conjunction with any of the above aspects and embodiments, the reactive moieties comprise an unsaturated group.

In some embodiments, which can be used in conjunction with any of the above aspects and embodiments, the reactive polymer is a multi-arm polymer that comprises three or more polymer arms linked to a core region, each arm comprising a lactone residue disposed between one of the hydrophilic polymer segments and one of the first reactive moieties, wherein the reactive moieties comprise an unsaturated group.

In some embodiments, which can be used in conjunction with the above embodiments, the core region comprises a polyol residue.

In some embodiments, which can be used in conjunction with the above embodiments, the hydrophilic polymer segments are selected from polyalkylene oxide segments, polyester segments, polyoxazoline segments, polydioxanone segments, and polypeptide segments.

In some embodiments, which can be used in conjunction with any of the above aspects and embodiments, each of the hydrophilic polymer segments contains between 10 and 1000 monomer residues.

In some embodiments, which can be used in conjunction with any of the above aspects and embodiments, the systems comprise a first composition that comprises the radiopaque polyamino compound in a first container and a second composition that comprises the reactive polymer in a second container, wherein the first container and the second container are independently selected from vials and syringe barrels. For example, the first container may be a syringe barrel, and the second container may be a vial, among other possibilities.

In some embodiments, which can be used in conjunction with any of the above aspects and embodiments, the systems further comprise a delivery device. For example, the delivery device may comprise a double barrel syringe, among other possibilities.

In some aspects, the present disclosure provides systems for forming a hydrogel that comprise, (i) a reactive polymer comprising a plurality of hydrophilic polymer segments and a plurality of reactive moieties and (ii) a radiopaque polyamino compound,

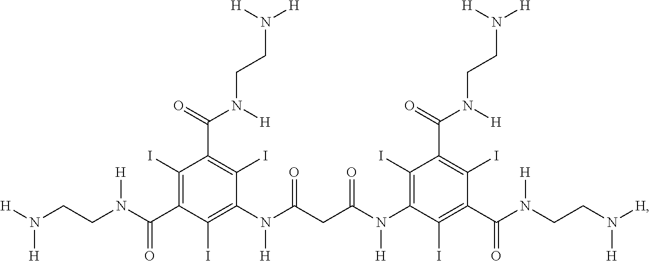

wherein the reactive moieties are reactive with the amino groups of the radiopaque polyamino compound.

In other aspects, the present disclosure pertains to crosslinked radiopaque hydrogels produced by a system in accordance with any of the above aspects and embodiments.

In some embodiments, the crosslinked radiopaque hydrogel may have a radiopacity that is greater than 100 Hounsfield units (HU).

In further aspects, the present disclosure pertains to methods of treatment comprising administering to a subject a mixture that comprises (i) a reactive polymer in accordance with any of the above aspects and embodiments and (ii) a radiopaque polyamino compound in accordance with any of the above aspects and embodiments, wherein the mixture is administered under conditions such that the amino groups of the radiopaque polyamino compound and the reactive moieties of the reactive polymer form covalent crosslinks after administration.

In some embodiments, the methods comprise administering to the subject a first fluid composition that comprises the radiopaque polyamino compound and the reactive polymer and a second fluid composition that comprises an accelerant that accelerates formation of the covalent crosslinks. In some of these embodiments, the first fluid composition and the second fluid composition are delivered using a double barrel syringe.

In various embodiments of the present disclosure, iodinated polyamino compounds are formed, which are radiopaque, are easy to synthesize, and are expected to be biologically well-tolerated.

In various embodiments of the present disclosure, iodinated polyamino compounds are formed, which have no covalent bonds, including ester bonds, that are susceptible to hydrolysis.

The above and other aspects, embodiments, features and benefits of the present disclosure will be readily apparent from the following detailed description.

### BRIEF DESCRIPTION OF THE DRAWINGS

FIG. 1 schematically illustrates the formation of a radiopaque polyamino compound from iodixanol by substituting hydroxyl groups of the iodixanol with primary amine groups, in accordance with an embodiment of the present disclosure.

FIG. 1A schematically illustrates the formation of a radiopaque polyamino compound from iodixanol by substituting hydroxyl groups of the iodixanol with primary amine groups, in accordance with another embodiment of the present disclosure.

FIG. 1B schematically illustrates the formation of a radiopaque polyamino compound from iodixanol by substituting hydroxyl groups of the iodixanol with primary amine groups, in accordance with yet another embodiment of the present disclosure.

FIG. 2 schematically illustrates the formation of a radiopaque polyamino compound from iodixanol by converting only a single hydroxyl group in each vicinal diol group of the iodixanol into an amino group, in accordance with an embodiment of the present disclosure.

FIG. 3 schematically illustrates the formation of a radiopaque polyamino compound from iodixanol by converting each vicinal diol group of the iodixanol into an aminomethyl group, in accordance with an embodiment of the present disclosure.

FIG. 4A schematically illustrates a process for the formation of acrylate-terminated eight-arm PEG, in accordance with an embodiment of the present disclosure.

FIG. 4B schematically illustrates a process for the formation of a hydroxybutyl-ester-terminated eight-arm PEG, in accordance with an embodiment of the present disclosure.

FIG. 5 schematically illustrates a process for the formation of a radiopaque crosslinked polymer network by the reaction of the radiopaque polyamino compound of FIGS. 1A-1B with an eight-arm PEG having reactive succinimidyl glutarate groups, in accordance with an embodiment of the present disclosure.

FIG. 6 schematically illustrates a delivery device, in accordance with an embodiment of the present disclosure.

FIG. 7 schematically illustrates a delivery device, in accordance with another embodiment of the present disclosure.

### DETAILED DESCRIPTION

In various aspects of the present disclosure, radiopaque polyamino compounds are formed from polyhydroxylated radiopaque compounds, many of which are widely used and have well-characterized biocompatibility profiles. Polyhydroxylated radiopaque compounds for use herein may contain two, three, four, five, six, seven, eight, nine, ten, eleven, twelve or more hydroxyl groups.

Particular examples of polyhydroxylated radiopaque compounds include iodine-containing polyhydroxylated compounds, several of which are commercially available, include the following, among others: iopromide,

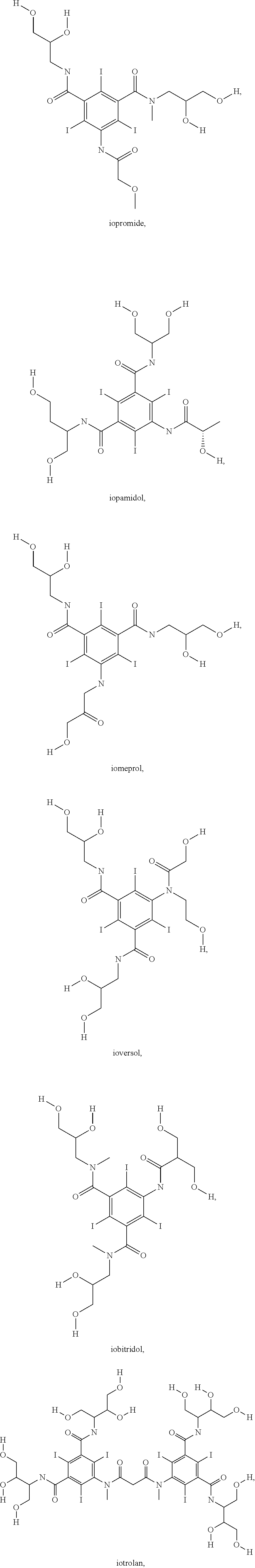

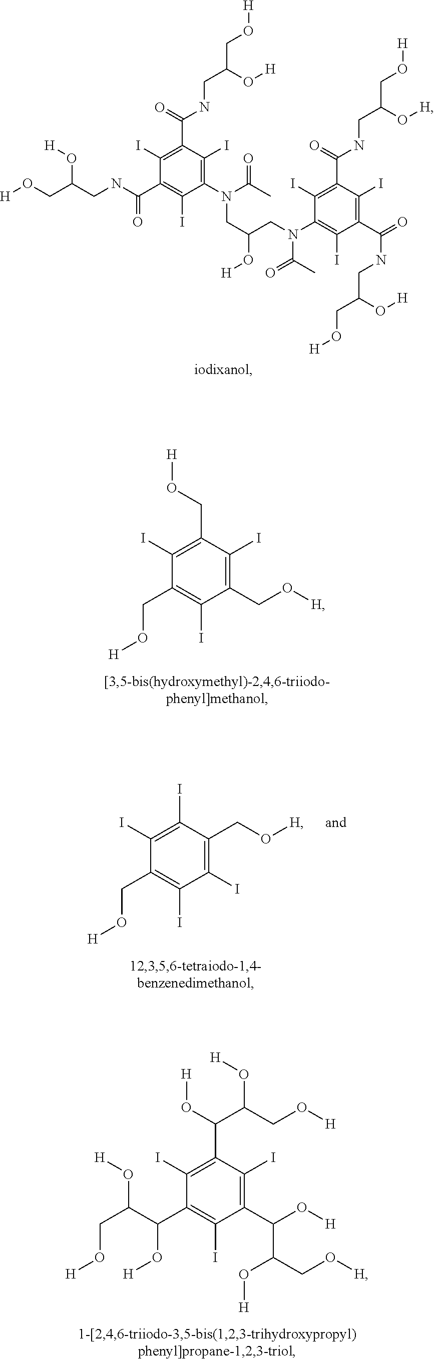

among others.

Polyhydroxylated radiopaque compounds for use herein include compounds that comprise one or more hydroxy-substituted iodinated aromatic groups, such as those set forth above among many others. In some embodiments, compounds that comprise one or more hydroxy-substituted iodinated aromatic groups include compounds that contain at least one aromatic group that is substituted with one or more (e.g., one, two, three, four, five, six, seven, eight, nine, or more) iodine groups and one or more (e.g., one, two, three, four, five, six, seven, eight, nine, or more) hydroxy-containing groups, which may be selected, for example, from hydroxy groups and hydroxyalkyl groups (e.g., hydroxyalkyl groups containing (i) one hydroxy group, two hydroxy groups, three hydroxy groups, four hydroxy groups, or more and (ii) one carbon, two carbons, three carbons, four carbons, five carbons, six carbons, or more).

In some embodiments, compounds that comprise one or more hydroxy-substituted iodinated aromatic groups include compounds that contain at least one monocyclic or multicyclic aromatic group that is substituted with one or more (e.g., one, two, three, four, five, six, seven, eight, nine, or more) iodine groups and one or more (e.g., one, two, three, four, five, six, seven, eight, nine, or more) hydroxy-containing groups. The hydroxy-containing groups may be linked to the monocyclic aromatic or multicyclic aromatic groups directly or through a linkage that contains one or more amine groups, one or more carbonyl groups, one or more amide groups, one or more ether groups, and combinations of such groups, among others. Examples of monocyclic and multicyclic aromatic groups include benzene groups, naphthalene groups, anthracene groups, phenanthrene groups, and tetracene groups, among others. Examples of hydroxy-containing groups include hydroxyl groups and/or C₁-C₆-hydroxyalkyl groups (e.g., C₁-C₆-monohydroxyalkyl groups, C₁-C₆-dihydroxyalkyl groups, C₁-C₆-trihydroxyalkyl groups, C₁-C₆-tetrahydroxyalkyl groups, C₁-C₆-pentahydroxyalkyl groups, etc.). In certain embodiments, the hydroxyl-containing groups are hydroxyalkyl groups that contain a vicinal diol group, such as a vicinal C₁-C₆-dihydroxyalkyl group (i.e., C₁-C₆-dihydroxyalkyl groups in which the two hydroxyl groups are positioned on adjacent carbon atoms), among other possibilities. A particular example of a vicinal dihydroxy-C₁-C₆-alkyl group is a 2,3-dihydroxypropyl group, in which the 2-hydroxyl group is a secondary hydroxyl group the 3-hydroxyl group is a primary hydroxyl group.

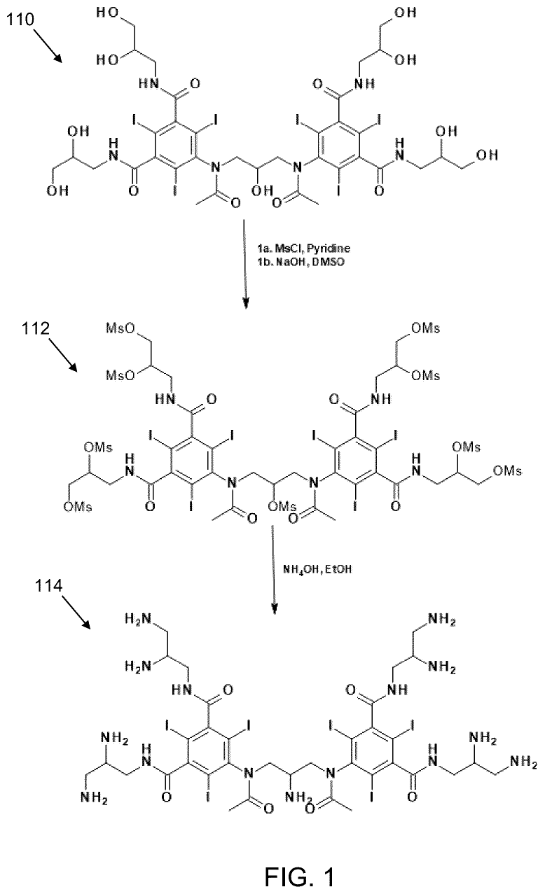

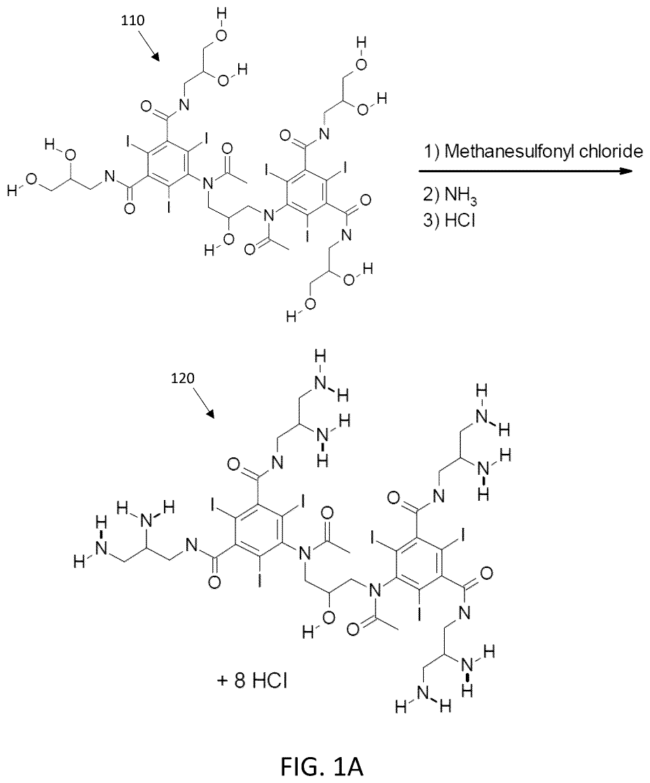

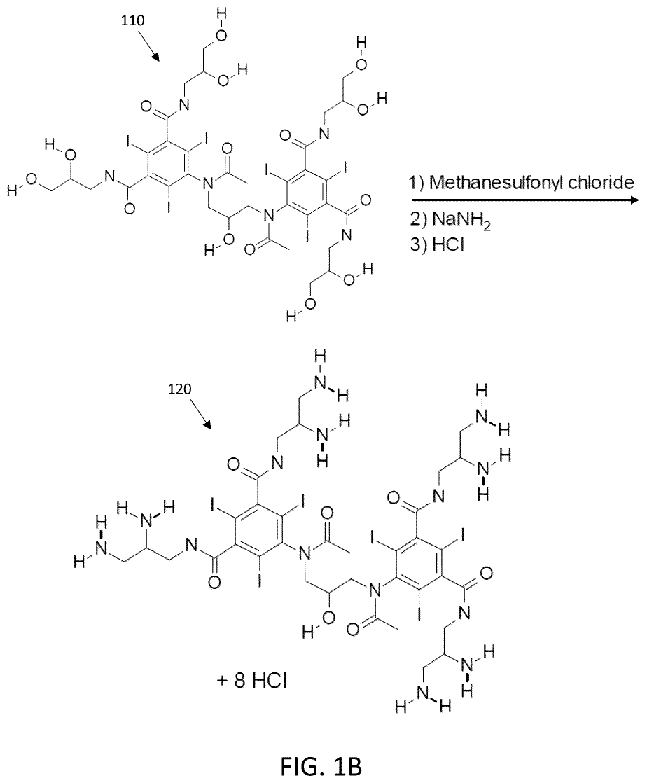

In some embodiments, every hydroxyl group is converted into an amino group. For example, with reference now to FIG. 1, 5,5′-((2-hydroxytrimethylene)bis(acetylimino))bis(N,N′-bis(2,3-dihydroxypropyl)-2,4,6-triiodoisophthalamide), also known as iodixanol (110), which is known to be biocompatible and is used in patients undergoing computed tomography (CT), is first reacted with methanesulfonyl chloride (MsCl) in pyridine followed by the addition of NaOH in dimethyl sulfoxide (DMSO) to form an intermediate compound (112) in which each of the hydroxyl groups of the iodixanol is converted into a methanesulfonate group (designated as OMs). The methanesulfonate groups are then reacted with ammonium hydroxide in ethanol to convert the methanesulfonate groups to amino groups, thereby forming iodixanol in which the hydroxy groups are replaced by amino groups, or 5,5′-((2-aminotrimethylene)bis(acetylimino))bis(N,N′-bis(2,3-diaminopropyl)-2,4,6-triiodoisophthalamide) (114). While all nine of the hydroxyl groups are replaced by amino groups in FIG. 1, in other embodiments only a portion of the hydroxyl groups (e.g., two, three, four, five, six, seven, or eight hydroxyl) may be replaced.

In another embodiment, the methanesulfonate groups may be reacted with sodium azide in order to replace every alcohol group with an azide group, after which the azide groups are reduced to amino groups. In yet another alternative embodiment, iodixanol is treated with 4-toluene sulfonyl chloride (tosyl chloride) in pyridine thereby replacing the hydroxyl groups with tosyl groups, followed by reaction with ammonium hydroxide to replace the chloride groups with amine groups.

In a further embodiment, with reference now to FIG. 1A, iodixanol (110) is first treated with methanesulfonyl chloride (CAS #124-63-0) to form an intermediate compound in which hydroxyl groups of the iodixanol are converted into methanesulfonate groups. The methanesulfonate groups are then reacted with ammonia, followed by treatment with hydrochloric acid to convert the methanesulfonate groups to amino groups (specifically, the ammonium salt form), thereby forming iodixanol in which the hydroxy groups are replaced by amino groups, or 5,5′-((2-hydroxytrimethylene)bis(acetylimino))bis(N,N′-bis(2,3-diaminopropyl)-2,4,6-triiodoisophthalamide) (120). In FIG. 1A, as well as FIG. 1B to follow, all of the hydroxyl groups of the iodixanol are illustrated as being converted to amino groups, except for the hydroxyl substituent of the central trimethylene group of the iodixanol molecule, which may be difficult to covert as a result of steric effects. However, under specific conditions, such as a long period of reaction time, all nine hydroxyl groups may be converted. Under other specific conditions, such as with a controlled feeding stock, only the four primary nine hydroxyl groups may be converted.

As an alternative, and with reference now to FIG. 1B, iodixanol (110), is first treated with methanesulfonyl chloride (CAS #124-63-0) to form an intermediate compound in which the hydroxyl groups of the iodixanol are converted into methanesulfonate groups. The methanesulfonate groups are then reacted with sodium amide, NaNH₂ (also known as sodium azanide), followed by treatment with hydrochloric acid to convert the methanesulfonate groups to amino groups, thereby forming 5,5′-((2-hydroxytrimethylene)bis(acetylimino))bis(N,N′-bis(2,3-diaminopropyl)-2,4,6-triiodoisophthalamide (120).

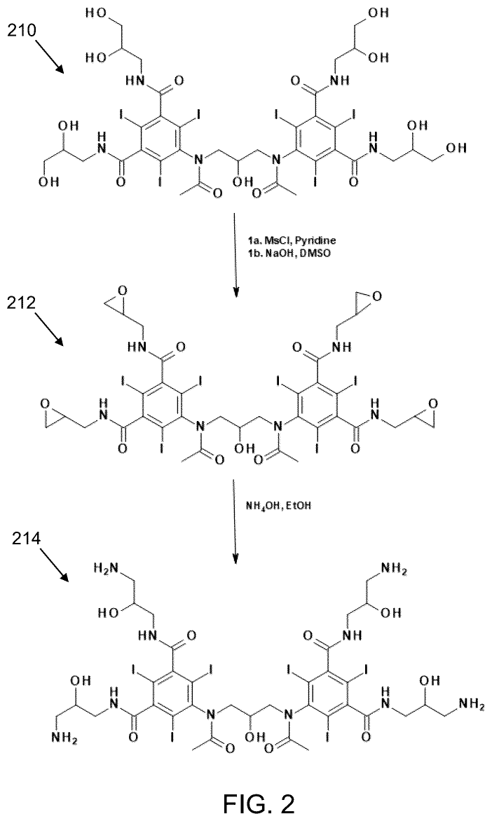

In some embodiments, only a single hydroxyl group of each vicinal diol group is converted into an amino group. For example, with reference now to FIG. 2, iodixanol (210) is reacted with methanesulfonyl chloride (MsCl) in pyridine followed by treatment with NaOH in dimethyl sulfoxide (DMSO) to form an intermediate compound (212) in which an epoxide group is formed at the site of every vicinal diol group. See, e.g., Anthony Clouet, et al., Adv. Synth. Catal. 2004, 346, 1195-1204. In contrast to FIG. 1, the 2,3-diol is allowed to cyclize to an epoxide by ring closure of the methanesulfonyl appended alcohol with its corresponding proximal hydroxyl group. The epoxide groups are then reacted with ammonium hydroxide in ethanol to convert the epoxide groups into hydroxyaminoethyl groups (—CHOHCH₂NH₂ groups), thereby forming iodixanol in which the primary hydroxy group of each vicinal diol group is replaced by an amino group, or 5,5′-((2-hydroxytrimethylene)bis(acetylimino))bis(N,N′-bis(2-hydroxy-3-aminopropyl)-2,4,6-triiodoisophthalamide) (214).

In an alternative embodiment iodixanol is treated with 4-toluene sulfonyl chloride, rather than methanesulfonyl chloride, to form the corresponding tosyl-appended alcohols which cyclize to the same epoxide (212).

In further embodiments, radiopaque polyamino compounds are formed from polyhydroxylated radiopaque compounds by converting vicinal diol groups (—CHOHCH₂OH groups) of polyhydroxylated radiopaque compounds into aminomethyl groups (—CH₂NH₂ groups).

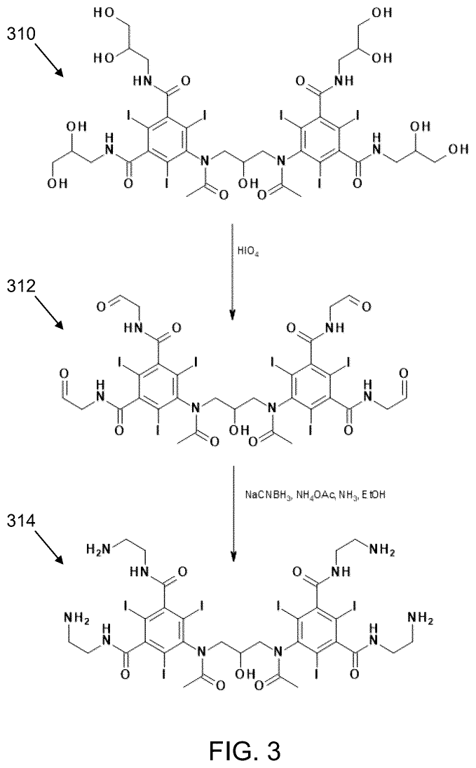

In one embodiment illustrated in FIG. 3, iodixanol (310) is first reacted with periodic acid (HIO₄) to cleave vicinal diol groups and form aldehydes. The result is an intermediate compound (312) in which an aldehyde group is created at the site previously occupied by each vicinal diol group. The aldehyde groups are then reacted with sodium cyanoborohydride, ammonia, and ammonium acetate in ethanol leading to metal hydride/ammonia mediated reductive amination of the aldehyde groups to form amine groups. See, e.g., Emma M. Dangerfield et al., J. Org. Chem. 2010, 75, 5470-5477. The result is a compound in which each vicinal diol pair of the iodixanol is replaced with a primary amine group, specifically, 5,5′-((2-hydroxytrimethylene)bis(acetylimino))bis(N,N′-bis(2-aminoethyl)-2,4,6-triiodoisophthalamide) (314).

Using these and other techniques, a variety of radiopaque polyamino compounds may be formed from polyhydroxylated radiopaque compounds by converting all or a portion of the hydroxyl groups of the polyhydroxylated radiopaque compounds to amino groups or by converting all or a portion of the vicinal diol groups of the polyhydroxylated radiopaque compounds to aminomethyl groups. Radiopaque polyamino compounds in accordance with the present disclosure may contain two, three, four, five, six, seven, eight, nine, ten, eleven, twelve, or more amino groups.

Additional examples of radiopaque polyamino compounds include the following, among others: iopromide, wherein at least a portion of the hydroxyl groups, specifically, four hydroxyl groups, have been substituted by amino groups, with the result being N,N′-Bis(2,3-diaminopropyl)-2,4,6-triiodo-5-(2-methoxyacetamido)-N-methylisophthalamide,

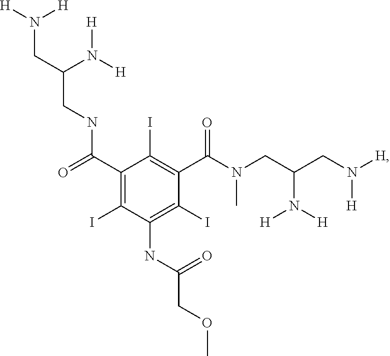

iopamidol, where at least a portion of the hydroxyl groups, specifically, five hydroxyl groups, have been substituted by amino groups, with the result being N,N′-bis(2-amino-1-(aminomethyl)ethyl)-2,4,6-triiodo-5-lactamidoisophthalamide

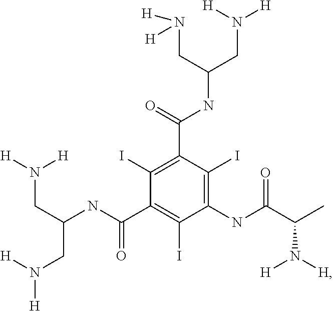

iomeprol, wherein at least a portion of the hydroxyl groups, specifically, five hydroxyl groups, have been substituted by amino groups, with the result being N,N′-bis(2,3-aminopropyl)-5-(2-amino-N-methylacetamido)-2,4,6-triiodoisophthalamide,

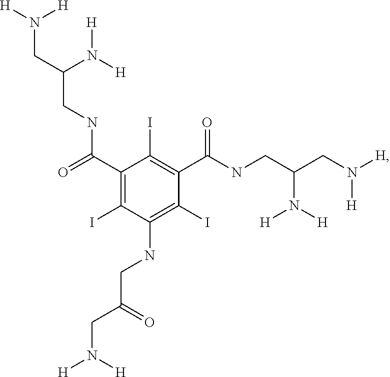

ioversol, wherein at least a portion of the hydroxyl groups, specifically, six hydroxyl groups have been substituted by amino groups, with the result being N,N′-bis(2,3-diaminopropyl)-5-(2-amino-N-(2-amino) acetamido)-2,4,6-triiodoisophthalamide,

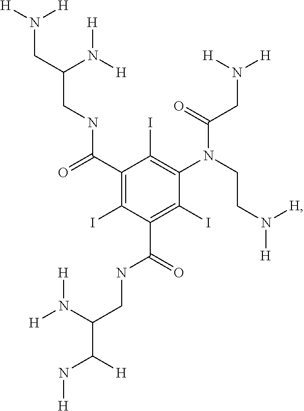

iobitridol, wherein at least a portion of the hydroxyl groups, specifically, six hydroxyl groups, have been substituted by amino groups, with the result being N,N′-bis(2,3-diaminopropyl)-5-((5-amino-2-(aminomethyl)-1-oxopropyl)amino)-2,4,6-triiodo-N,N′-dimethyl-1,3-benzenedicarboxamide,

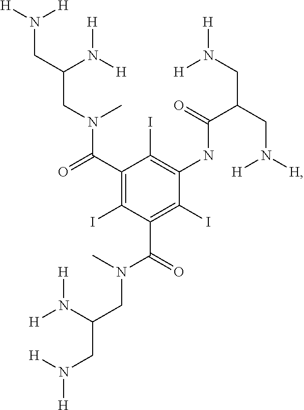

iodixanol, wherein at least a portion of the hydroxyl groups, specifically, eight out of nine hydroxyl groups, have been substituted by amino groups, with the result being 5,5′-((2-hydroxytrimethylene)bis(acetylimino))bis(N,N′-bis(2,3-diaminopropyl)-2,4,6-triiodoisophthalamide,

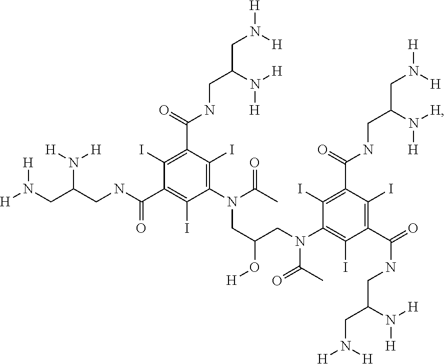

and iotrolan, wherein at least a portion of the hydroxyl groups, specifically, twelve hydroxyl groups, have been substituted by amino groups, with the result being 5,5′-((1,3-dioxo-1,3-propanediyl)bis(methylimino))bis(N,N′-bis(2,3-diamino-1-(aminomethyl)propyl)-2,4,6-triiodo-1,3-benzenedicarboxamide,

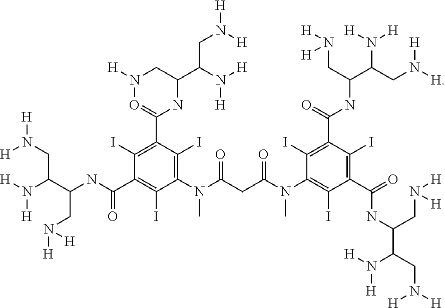

In some embodiments, the radiopaque polyamino compound need not be formed from a radiopaque polyhydroxylated compound. One example of such a radiopaque polyamino compound is CA⁴⁺:

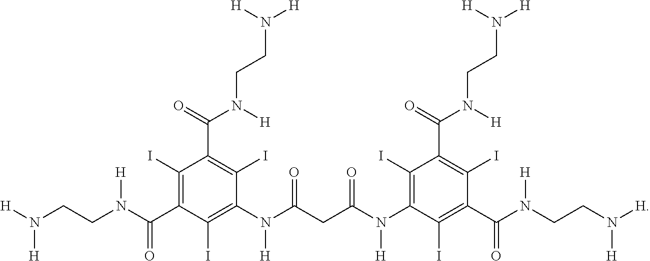

Radiopaque polyamino compounds in accordance with the present disclosure include compounds that comprise one or more amino-substituted iodinated aromatic groups, including the radiopaque polyamino compounds set forth above, among many others. In some embodiments, compounds that comprise one or more amino-substituted iodinated aromatic groups include compounds that contain at least one aromatic group that is substituted with one or more (e.g., one, two, three, four, five, six, seven, eight, nine, or more) iodine groups and one or more (e.g., one, two, three, four, five, six, seven, eight, nine, or more) amino-containing groups, which may be selected, for example, from amino groups, aminoalkyl groups (e.g., aminoalkyl groups containing (i) one amino group, two amino groups, three amino groups, four amino groups, or more and (ii) one carbon, two carbons, three carbons, four carbons, five carbons, six carbons, or more), and hydroxyaminoalkyl groups (e.g., hydroxyaminoalkyl groups containing (i) one amino group, two amino groups, three amino groups, four amino groups, or more, (ii) one hydroxyl group, two hydroxyl groups, three hydroxyl groups, four hydroxyl groups, or more, and (iii) one carbon, two carbons, three carbons, four carbons, five carbons, six carbons, or more).

In some embodiments, compounds that comprise one or more amino-substituted iodinated aromatic groups include compounds that contain at least one monocyclic or multicyclic aromatic group that is substituted with one or more (e.g., one, two, three, four, five, six, seven, eight, nine, or more) iodine groups and one or more (e.g., one, two, three, four, five, six, seven, eight, nine, or more) amino-containing groups. The amino-containing groups may be directly linked to the one or more monocyclic aromatic or multicyclic aromatic groups or through a linkage that contains one or more amine groups, one or more carbonyl groups, one or more amide groups, one or more ether groups, and combinations of such groups. Examples of monocyclic and multicyclic aromatic groups include benzene groups, naphthalene groups, anthracene groups, phenanthrene groups, and tetracene groups, among others. Examples of amino-containing groups include C₁-C₆-aminoalkyl groups. Particular examples of C₁-C₆-aminoalkyl groups include 2-aminoethyl and 2,3-diaminopropyl groups. Examples of amino-containing groups also include C₁-C₆-hydroxyaminoalkyl groups. In certain embodiments, C₁-C₆-hydroxyaminoalkyl groups are formed in which a hydroxyl group and an amino group are positioned on adjacent carbon atoms. A particular example of a C₁-C₆-hydroxyaminoalkyl group is a 2-hydroxy-3-aminopropyl group.

In some aspects of the present disclosure, crosslinked radiopaque hydrogels are provided that comprises a crosslinked reaction product of (a) a radiopaque polyamino compound in accordance with the present disclosure and (b) a reactive polymer comprising reactive moieties that are reactive with the amino groups of the radiopaque polyamino compound.

Reactive polymers for use in the present disclosure include reactive multi-arm polymers that comprise a plurality of polymer arms linked to a core region, wherein the polymer arms comprise a hydrophilic polymer segment. In some embodiments, a first end of the hydrophilic polymer segment is covalently linked to the core region and a reactive moiety is covalently linked to a second end (opposite end) of the hydrophilic polymer segment.

In some embodiments, the reactive moiety is covalently linked to the second end of the hydrophilic polymer segment through a cyclic anhydride residue or a lactone residue. For example, the second end of the hydrophilic polymer segment may be covalently linked to a first end of the cyclic anhydride or lactone residue and the reactive moiety may be covalently linked to a second end of the cyclic anhydride or lactone residue.

Reactive polymers in accordance with the present disclosure include multi-arm polymers having from 3 to 100 arms, for example ranging anywhere from 3 to 4 to 5 to 6 to 7 to 8 to 9 to 10 to 11 to 12 to 15 to 20 to 25 to 50 to 75 to 100 arms (in other words, ranging between any two of the preceding numerical values).

Reactive moieties include reactive moieties that comprise electrophilic groups and reactive moieties that comprise unsaturated groups.

Electrophilic groups may be selected, for example, from cyclic imide ester groups, such as succinimide ester groups,

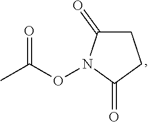

maleimide ester groups, glutarimide ester groups, diglycolimide ester groups, phthalimide ester groups, and bicyclo[2.2.1]hept-5-ene-2,3-dicarboxylic acid imide ester groups,

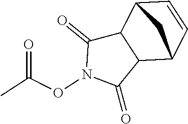 [SMILES: CC(=O)ON1C(=O)C2C(C1=O)[C@H]1C=C[C@@H]2C1]

imidazole ester groups, imidazole carboxylate groups and benzotriazole ester groups, among other possibilities.

Unsaturated groups may be selected, for example, from unsaturated end groups having double carbon-carbon bonds such as acrylate ester groups and unsaturated end groups having triple carbon-carbon bonds such as propiolate ester groups.

The electrophilic or unsaturated groups may be linked to the hydrophilic polymer segment through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others. In certain embodiments, the linking moiety comprises a hydrolysable ester group.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 5 and 1000 monomer units or more, for example, ranging anywhere from 5 to 10 to 20 to 50 to 100 to 200 to 500 to 1000 to 2000 monomer units.

In certain embodiments, the core region comprises a residue of a polyol comprising three or more hydroxyl groups, which is used to form the polymer arms. For example, the core region may comprise a residue of a polyol that contains from 3 to 100 hydroxyl groups, for example ranging anywhere from 3 to 4 to 5 to 6 to 7 to 8 to 9 to 10 to 11 to 12 to 15 to 20 to 25 to 50 to 75 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, tetrapentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative polyols also include calixarenes such as, for example, calix[n] arenes where n is 4, 5, 6, 7, 8, 9, 10, 11, 12, or more, among other possibilities.

Illustrative polyols further include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 4 to 5 to 6 to 8 to 10 to 25 to 50 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three 0 atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The Ts cage-like silicon-oxygen cores are widely studied and have the formula [RSiO_{3/2}]₈, or equivalently R₈Si₈O₁₂. Such a structure is shown here:

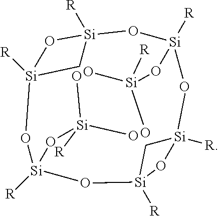

In the present disclosure, the R groups comprise the polymer arms described herein.

Reactive multi-arm polymers in accordance with the present disclosure can be formed from hydroxyl-terminated precursor multi-arm polymers having arms that comprise one or more hydroxyl end groups.

In some of these embodiments, the hydroxyl-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxyl-terminated precursor multi-arm hydrophilic polymer under basic conditions to form a carboxylic-acid-terminated precursor polymer comprising a carboxylic acid end group that is linked to a hydrophilic polymer segment through a hydrolysable ester group.

A reactive moiety may then be linked to the carboxylic-acid-terminated precursor polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl'propyl)carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

Reactive multi-arm polymers having reactive moieties that comprise unsaturated groups can also be formed from hydroxy-terminated precursor multi-arm polymers having arms that comprise one or more hydroxyl end groups.

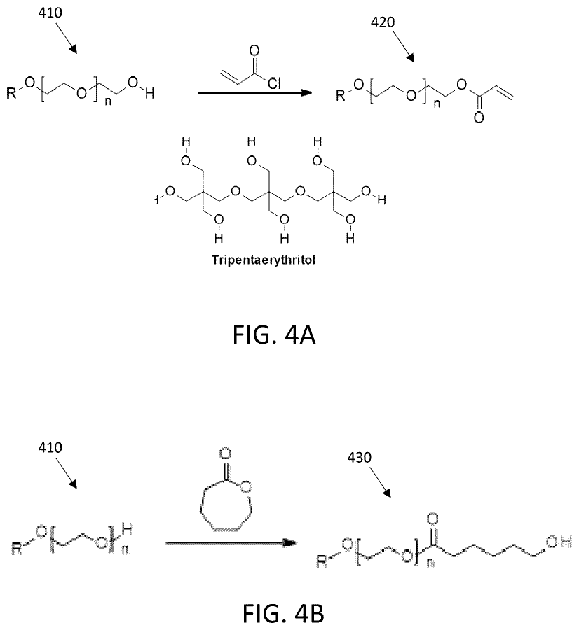

In some embodiments, an unsaturated moiety may be linked a hydroxyl-terminated precursor multi-arm polymer. In a particular example, a reactive multi-arm polymer may be formed by reacting acryloyl chloride with hydroxyl end groups of a hydroxyl-terminated precursor multi-arm polymer, thereby forming unsaturated acrylate ester groups at the sites previously occupied by the hydroxyl groups. In one embodiment shown in FIG. 4A, a reactive multi-arm polymer, which comprises a core region and a plurality of polyethylene oxide (PEO) arms having reactive acrylate end groups is formed by reacting acryloyl chloride with hydroxyl end groups of a hydroxyl-terminated multi-arm polymer having a core region that comprises a polyol residue and multiple hydroxyl-terminated polyethylene oxide arms. In particular, a commercially available hydroxyl-terminated 8-arm PEG (410) having a tripentaerythritol polyol residue core where n ranges from 30 to 140 (only one polymer arm is shown; the tripentaerythritol polyol residue and the remaining seven arms are represented by R), is reacted with acryloyl chloride to create an acrylate-terminated 8-arm PEO (420). Acrylate-terminated 8-arm PEO (also referred to acrylate-terminated 8-arm PEG) having a tripentaerythritol residue core and acrylate-terminated 4-arm PEO (also referred to an acrylate-terminated 4-arm PEG) having a pentaerythritol residue core are also available from JenKem Technology USA (Plano, Texas, USA).

In some embodiments, it is desirable to include a hydrolysable ester group in order to enhance the biodegradability of the reactive multi-arm polymer via hydrolysis. For example, hydroxyl end groups of a hydroxyl-terminated precursor multi-arm polymer may be reacted in a ring-opening reaction with a cyclic-ester-containing compound, specific examples of which include lactone compounds such as β-propiolactone, γ-butyrolactone, δ-valerolactone, and ε-caprolactone, among other lactones, thereby forming a multi-arm polymer having hydroxyalkyl end groups, for example, C₁-C₆-hydroxyalkyl end groups, which are linked to the polymer arm through an ester group. In other words, a hydroxyl-terminated multi-arm polymer is formed in which polymer arms are end capped with a hydroxyalkyl ester group, such as a hydroxymethyl ester group, a hydroxyethyl ester group, a hydroxypropyl ester group, a hydroxybutyl ester group, etc. In a particular embodiment shown in FIG. 4B, terminal hydroxyl groups of a hydroxyl-terminated multi-arm polymer, for example, a hydroxyl-terminated 8-arm PEG (410) having a core region that comprises a polyol residue, for example, a tripentaerythritol residue, and multiple hydroxyl-terminated polyethylene oxide arms, for example, eight hydroxyl-terminated polyethylene oxide arms (only one polymer arm is shown; the polyol residue core and the remaining polymers arms are represented by R), where n ranges from 30 to 140, are reacted with a cyclic-ester-containing compound, in particular, ε-caprolactone, in a ring-opening reaction forming a hydroxybutyl-ester-end-capped multi-arm PEG (430). This hydroxyl-terminated polymer may then be reacted with acryloyl chloride as described above (and shown in FIG. 4A), thereby forming unsaturated acrylate ester groups at the sites previously occupied by the hydroxyl groups.

As previously noted, in some aspects, the present disclosure provides crosslinked radiopaque hydrogels that comprise a crosslinked reaction product of (a) a radiopaque polyamino compound in accordance with the present disclosure and (b) a reactive polymer comprising reactive moieties that are reactive with amino groups of the radiopaque polyamino compound.

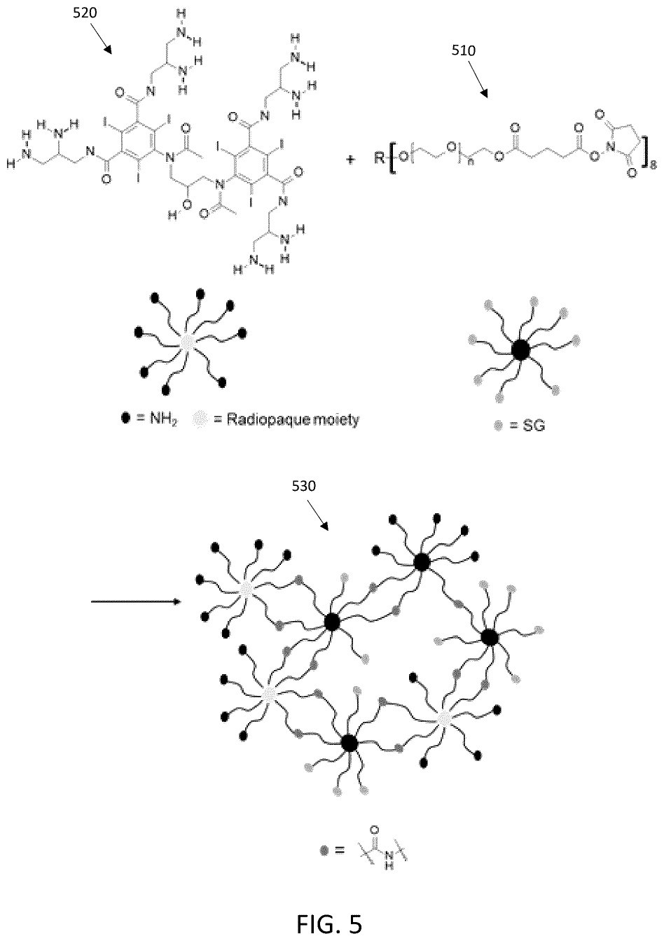

For example, in one specific embodiment shown in FIG. 5, a crosslinking reaction is conducted between a radiopaque polyamino compound, 5,5′-((2-hydroxytrimethylene)bis(acetylimino))bis(N,N′-bis(2,3-diaminopropyl)-2,4,6-triiodoisophthalamide) (520), which is iodixanol in which all but one of the hydroxy groups are replaced by amino groups as described in conjunction with FIGS. 1A-1B, and a reactive polymer having reactive moieties that comprise reactive cyclic imide ester groups, specifically an eight-arm PEG with reactive succinimidyl glutarate (SG) end groups (510), having a core region that comprises a polyol residue, specifically a tripentaerythritol polyol residue or a hexaglycerol polyol residue, where R represents the tripentaerythritol or hexaglycerol residue and n ranges from 30 to 140. The reactive polymer (510) and the radiopaque polyamino compound (520) are combined under conditions such that the succinimidyl ester groups of the reactive polymer (510) react with the amino groups of the radiopaque polyamino compound (520) to form amide bonds, accompanied by the release of N-hydroxysuccinimide, with the result being a crosslinked polymer network (530).

In certain embodiments, reaction between the succinimidyl ester groups and the amino groups is conducted at slightly basic pH (e.g., having a pH value ranging from 7.4 to 11) where the amino groups of the radiopaque polyamino compound are deprotonated/neutrally charged and amide bond formation can occur spontaneously at room or body temperature.

In another specific embodiment, a radiopaque polyamino compound as described herein, for example, 5,5′-((2-hydroxytrimethylene)bis(acetylimino))bis(N,N′-bis(2,3-diaminopropyl)-2,4,6-triiodoisophthalamide) described in conjunction with FIGS. 1A-1B is reacted with a reactive hydrophilic polymer having reactive moieties that comprise unsaturated groups, for example, an eight-arm PEG with reactive acrylate ester groups having a core region that comprises a tripentaerythritol residue like that described above. The reactive hydrophilic polymer and the radiopaque polyamino compound may be combined under conditions such that the unsaturated groups of the reactive hydrophilic polymer react with the primary amine groups of radiopaque polyamino compound via Michael addition to form amide bonds, thereby forming a crosslinked polymer network. In certain embodiments, reaction between the unsaturated groups and the amino groups is conducted at slightly basic pH (e.g., having a pH value ranging from 7.4 to 11) where the amino groups of the radiopaque polyamino compound are deprotonated/neutrally charged and the Michael addition can occur spontaneously at room or body temperature.

It will be appreciated that the crosslinking density of the resulting crosslinked radiopaque hydrogels can be tuned, for example, (a) by varying the number of primary amine groups in the radiopaque polyamino compound, (b) by varying the number of arms of the reactive multi-arm polymer, or (c) both.

Such crosslinked radiopaque hydrogels may be formed in vivo (e.g., using a delivery device like that described below), or such crosslinked radiopaque hydrogels may be formed ex vivo and subsequently administered to a subject. Such crosslinked radiopaque hydrogels can be used in a variety of biomedical applications, including implants, medical devices, and pharmaceutical compositions.

In some embodiments, the crosslinked radiopaque hydrogel is visible under fluoroscopy. The crosslinked radiopaque hydrogel may have a radiopacity that is greater than 100 Hounsfield units (HU), beneficially ranging anywhere from 100 HU to 250 HU to 500 HU to 750 HU to 1000 HU or more (in other words, ranging between any two of the preceding numerical values), for example, when measured on bench-top micro CT systems such as XtremeCT from Scanco Medical (Wangen-BrUttisellen, Switzerland) or similar.

In some aspects of the present disclosure, a system is provided that comprises (a) a first composition that comprises a radiopaque polyamino compound as described herein and (b) a second composition that comprises a reactive polymer comprising reactive moieties as described herein, wherein the system is configured to deliver the reactive polymer and the radiopaque polyamino compound under conditions such that covalent crosslinks are formed between the reactive polymer and the radiopaque polyamino compound.

The first composition may be a first fluid composition comprising the radiopaque polyamino compound or a first dry composition that comprises the radiopaque polyamino compound, to which a suitable fluid such as water for injection, saline, etc. can be added to form a first fluid composition. In addition to the radiopaque polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the radiopaque polyamino compound with a second fluid comprising the reactive polymer. Upon mixing the first and second fluid compositions, the radiopaque polyamino compound crosslinks with the reactive polymer, forming a crosslinked product. The first and second fluid compositions may be combined form crosslinked radiopaque hydrogels, either in vivo or ex vivo.

In some embodiments, the radiopaque polyamino compound is initially combined with the reactive polymer under conditions where crosslinking between the reactive polymer and the radiopaque polyamino compound is suppressed (e.g., an acidic pH). Then, when crosslinking is desired, the conditions are changed such that crosslinking is increased (e.g., a change from an acidic pH to a basic pH, in some embodiments), leading to crosslinking between the radiopaque polyamino compound and the reactive polymer, thereby forming a crosslinked product. The first and second fluid compositions may be combined form crosslinked radiopaque hydrogels, either in vivo or ex vivo.

In some embodiments, the system comprises (a) a first composition that comprises a radiopaque polyamino compound as described hereinabove, (b) a second composition that comprises a reactive polymer as described hereinabove, and (c) a third composition, specifically, an accelerant composition, that contains an accelerant that is configured to accelerate a crosslinking reaction between the radiopaque polyamino compound and the reactive polymer.

The first composition may be a first fluid composition comprising the radiopaque polyamino compound that is buffered to an acidic pH or a first dry composition that comprises the radiopaque polyamino compound, to which a suitable fluid such as water for injection, saline, an acidic buffer solution, etc. can be added to form a first fluid composition comprising the radiopaque polyamino compound that is buffered to an acidic pH. In some embodiments, for example, the acidic buffering composition may comprise monobasic sodium phosphate, among other possibilities. The first fluid composition comprising the radiopaque polyamino compound may have a pH ranging, for example, from about 3 to about 5. In addition to the radiopaque polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the radiopaque polyamino compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the radiopaque polyamino compound that is buffered to an acidic pH and the second composition comprises a dry composition that comprises the reactive polymer. The first composition may then be mixed with the second composition to provide a prepared fluid composition that is buffered to an acidic pH and comprises the radiopaque polyamino compound and the reactive polymer. In a particular example, a syringe may be provided that contains the first fluid composition comprising the radiopaque polyamino compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the radiopaque polyamino compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8 to 12. In addition to the above, the fluid accelerant composition may further comprise additional agents, including those described below.

A prepared fluid composition that is buffered to an acidic pH and comprises the radiopaque polyamino compound and the reactive polymer as described above, and a fluid accelerant composition that is buffered to basic pH as described above, may be combined form crosslinked radiopaque hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (in addition to the radiopaque polyamino compound), for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a radiopaque polyamino compound as described herein and a second reservoir that contains a second fluid composition that comprises a reactive polymer as described herein, wherein the first and second fluid compositions form a crosslinked product upon mixing.

In some embodiments, the system may include a delivery device that comprises a first reservoir that contains a first fluid composition that comprises the radiopaque polyamino compound and the reactive polymer and is buffered to an acidic pH, such as the prepared fluid composition previously described, and a second reservoir that contains a second fluid composition, such as the fluid accelerant composition previously described.

During operation, the first fluid composition and the second fluid composition are dispensed from the first and second reservoirs and combined, whereupon the radiopaque polyamino compound and the reactive polymer and crosslink with one another to form a crosslinked radiopaque hydrogel.

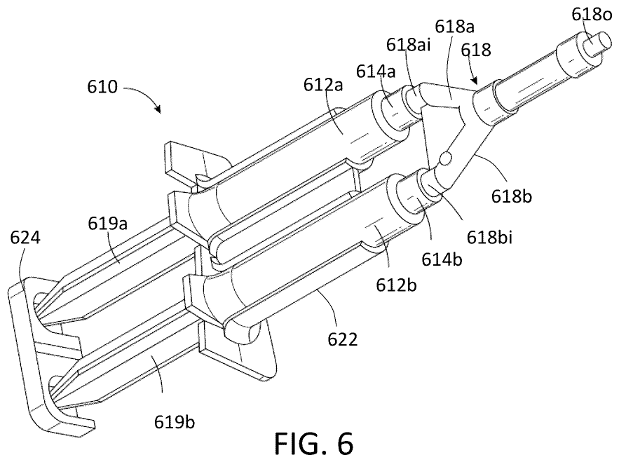

In particular embodiments, and with reference to FIG. 6, the system may include a delivery device 610 that comprises a double-barrel syringe, which includes a first barrel 612a having a first barrel outlet 614a, which first barrel contains a first fluid composition as described above, a first plunger 619a that is movable in the first barrel 612a, a second barrel 612b having a second barrel outlet 614b, which second barrel 612b contains a second fluid composition as described above, and a second plunger 619b that is movable in the second barrel 612b. In some embodiments, the device 610 may further comprise a mixing section 618 having a first mixing section inlet 618ai in fluid communication with the first barrel outlet 614a, a second mixing section inlet 618bi in fluid communication with the second barrel outlet 614b, and a mixing section outlet 618o. Also shown are a syringe holder 622 configured to hold the first and second syringe barrels 612a, 612b, in a fixed relationship and a plunger cap 624 configured to hold the first and second plungers 619a, 619b in a fixed relationship. In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a luer connector.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked radiopaque hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked radiopaque hydrogel is ultimately formed at the administration location.

During and/or after administration, the compositions of the present disclosure can be imaged using a suitable imaging technique. Typically, the imaging technique is an x-ray-based imaging technique, such as computerized tomography or x-ray fluoroscopy, or a near near-IR fluorescence spectrometry-based technique.

The first and second fluid compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions or fluid admixtures of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked radiopaque hydrogels may be in any desired form, including a slab, a cylinder, a coating, or a particle. In some embodiments, the crosslinked radiopaque hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked radiopaque hydrogel particles formed using the above and other techniques may vary widely in size, for example, having an average size ranging from 50 to 950 microns.

In addition to a crosslinked radiopaque hydrogel as described above, ex vivo crosslinked radiopaque hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the ex vivo crosslinked radiopaque hydrogel composition to a subject. Such systems may include one or more of the following: a syringe barrel, which may or may not contain a crosslinked radiopaque hydrogel composition as described herein; a vial, which may or may not contain a crosslinked radiopaque hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked radiopaque hydrogel composition may be provided in dry form (e.g., powder form) or in a form that is ready for injection, such as an injectable hydrogel form (e.g., a suspension of crosslinked radiopaque hydrogel particles).

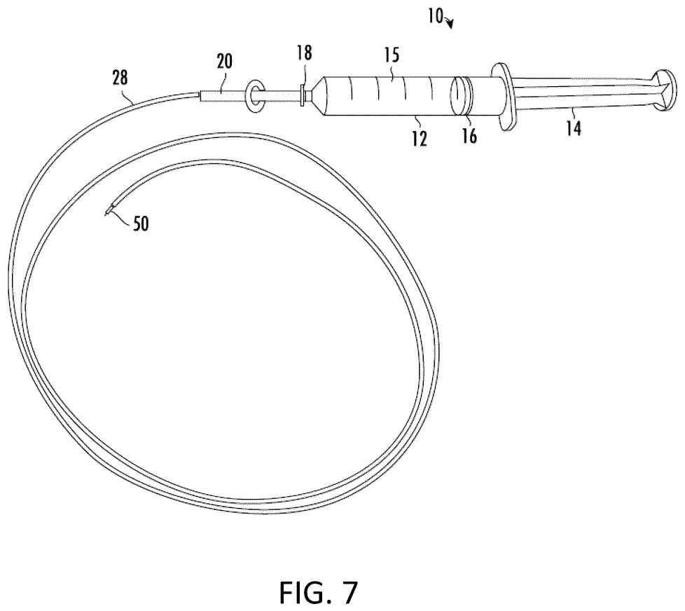

FIG. 7 illustrates a syringe 10 providing a reservoir for a crosslinked radiopaque hydrogel composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked radiopaque hydrogel composition 15 for injection through the needle 50.

The ex vivo crosslinked radiopaque hydrogel compositions described herein can be used for a number of purposes. For example, crosslinked radiopaque hydrogel compositions can be injected to provide spacing between tissues, crosslinked radiopaque hydrogel compositions can be injected (e.g., in the form of blebs) to provide fiducial markers, crosslinked radiopaque hydrogel compositions can be injected for tissue augmentation or regeneration, crosslinked radiopaque hydrogel compositions can be injected as a filler or replacement for soft tissue, crosslinked radiopaque hydrogel compositions can be injected to provide mechanical support for compromised tissue, crosslinked radiopaque hydrogel compositions be injected as a scaffold, and/or crosslinked radiopaque hydrogel compositions can be injected as a carrier of therapeutic agents in the treatment of diseases and cancers and the repair and regeneration of tissue, among other uses.

Ex vivo crosslinked radiopaque hydrogel compositions of the present disclosure may be used in a variety of medical procedures, including the following, among others: a procedure to implant a fiducial marker comprising a crosslinked radiopaque hydrogel, a procedure to implant a tissue regeneration scaffold comprising a crosslinked radiopaque hydrogel, a procedure to implant a tissue support comprising a crosslinked radiopaque hydrogel, a procedure to implant a tissue bulking agent comprising a crosslinked radiopaque hydrogel, a procedure to implant a therapeutic-agent-containing depot comprising a crosslinked radiopaque hydrogel, a tissue augmentation procedure comprising implanting a crosslinked radiopaque hydrogel, a procedure to introduce a crosslinked radiopaque hydrogel between a first tissue and a second tissue to space the first tissue from the second tissue.

Ex vivo crosslinked radiopaque hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Ex vivo crosslinked radiopaque hydrogel compositions in accordance with the present disclosure include lubricious compositions for medical applications, compositions for therapeutic agent release (e.g., by including one or more therapeutic agents in a matrix of the crosslinked radiopaque hydrogel), and implants (which may be formed ex vivo or in vivo) (e.g., compositions for use as tissue markers, compositions that act as spacers to reduce side effects of off-target radiation therapy, cosmetic compositions, etc.).

During and/or after administration, the ex vivo crosslinked radiopaque hydrogel compositions of the present disclosure can be imaged using a suitable imaging technique. Typically, the imaging technique is an x-ray-based imaging technique, such as computerized tomography or x-ray fluoroscopy, or a near near-IR fluorescence spectrometry-based technique.

1. A system for forming a hydrogel that comprises (a) a radiopaque polyamino compound and (b) a reactive polymer comprising a plurality of hydrophilic polymer segments and a plurality of reactive moieties, wherein the reactive moieties are reactive with amino groups of the radiopaque polyamino compound, and wherein the radiopaque polyamino compound is produced by a method that comprises: (i) converting hydroxyl groups of a polyhydroxylated radiopaque compound to amino groups, (ii) converting vicinal diol groups (—CHOHCH₂OH groups) of a polyhydroxylated radiopaque compound into hydroxyaminoethyl groups (—CHOHCH₂NH₂ groups), or (iii) converting vicinal diol groups of a polyhydroxylated radiopaque compound into aminomethyl groups (—CH₂NH₂ groups).

2. The system of claim 1, wherein the polyhydroxylated radiopaque compound comprises at least one hydroxy-substituted iodinated aromatic group.

3. The system of claim 2, wherein the at least one hydroxy-substituted iodinated aromatic group comprises a monocyclic or multicyclic aromatic group that is substituted with a plurality of iodine groups and a plurality of hydroxyalkyl-containing groups.

4. The system of claim 3, wherein the plurality of hydroxyalkyl-containing groups comprises C₂-C₆-hydroxyalkyl groups.

5. The system of claim 4, wherein the C₂-C₆-hydroxyalkyl groups comprise at least one vicinal diol group.

6. The system of claim 3, wherein the plurality of hydroxyalkyl-containing groups comprises at least one hydroxyalkyl-containing group that is linked to the monocyclic or multicyclic aromatic group through an amide-containing linkage.

7. The system of claim 1, wherein the polyhydroxylated radiopaque compound is selected from iopromide, iopamidol, iomeprol, ioversol, iobitridol, iotrolan, iodixanol, [3,5-bis(hydroxymethyl)-2,4,6-triiodo-phenyl]methanol, 1 2,3,5,6-tetraiodo-1,4-benzenedimethanol, and 1-[2,4,6-triiodo-3,5-bis(1,2,3-trihydroxypropyl)phenyl]propane-1,2,3-triol.

8. The system of claim 1, wherein the radiopaque polyamino compound comprises at least one amino-substituted iodinated aromatic group.

9. The system of claim 8, wherein the at least one amino-substituted iodinated aromatic group comprises a monocyclic or multicyclic aromatic group that is substituted with a plurality of iodine groups and a plurality of amino-containing groups.

10. The system of claim 9, wherein the plurality of amino-containing groups comprises aminoalkyl groups that comprise one or more amino groups and one or more carbon atoms.

11. The system of claim 10, wherein the aminoalkyl groups comprise at least one C₂-C₆-aminoalkyl group that comprises two amino groups positioned on adjacent carbon atoms and/or or wherein the aminoalkyl groups comprises at least one C₂-C₆-aminoalkyl group that comprises a single terminal amino group.

12. The system of claim 9, wherein the plurality of amino-containing groups comprises at least one hydroxyaminoalkyl group that comprises one or more amino groups, one or more hydroxyl groups and one or more carbon atoms.

13. The system of claim 12, wherein the at least one hydroxyaminoalkyl group comprises at least one C₂-C₆-hydroxyaminoalkyl group that comprises a hydroxyl group and an amino group positioned on adjacent carbon atoms.

14. The system of claim 9, wherein the plurality of amino-containing groups comprises at least one amino-containing group that is linked to the monocyclic or multicyclic aromatic group through an amide-containing linkage.

15. The system of claim 1, wherein the reactive polymer is a multi-arm polymer that comprises three or more polymer arms linked to a core region, each arm comprising one of the hydrophilic polymer segments and one of the reactive moieties that are reactive with the amino groups of the radiopaque polyamino compound to form covalent crosslinks.

16. The system of claim 15, wherein the core region comprises a polyol residue and/or wherein the hydrophilic polymer segments are selected from polyalkylene oxide segments, polyester segments, polyoxazoline segments, polydioxanone segments, and polypeptide segments.

17. The system of claim 1, comprising a first composition that comprises the radiopaque polyamino compound in a first container and a second composition that comprises the reactive polymer in a second container, wherein the first container and the second container are independently selected from vials and syringe barrels.

18. The system of claim 17, wherein the first container is a syringe barrel, and the second container is a vial.

19. A crosslinked radiopaque hydrogel produced by system that comprises (a) a radiopaque polyamino compound and (b) a reactive polymer comprising a plurality of hydrophilic polymer segments and a plurality of reactive moieties, wherein the reactive moieties are reactive with amino groups of the radiopaque polyamino compound, and wherein the radiopaque polyamino compound is produced by a method that comprises: (i) converting hydroxyl groups of a polyhydroxylated radiopaque compound to amino groups, (ii) converting vicinal diol groups (—CHOHCH₂OH groups) of a polyhydroxylated radiopaque compound into hydroxyaminoethyl groups (—CHOHCH₂NH₂ groups), or (iii) converting vicinal diol groups of a polyhydroxylated radiopaque compound into aminomethyl groups (—CH₂NH₂ groups).

20. A method of treatment comprising administering to a subject a mixture that comprises (a) a radiopaque polyamino compound and (b) a reactive polymer comprising a plurality of hydrophilic polymer segments and a plurality of reactive moieties, wherein the reactive moieties are reactive with amino groups of the radiopaque polyamino compound, and wherein the radiopaque polyamino compound is produced by a method that comprises: (i) converting hydroxyl groups of a polyhydroxylated radiopaque compound to amino groups, (ii) converting vicinal diol groups (—CHOHCH₂OH groups) of a polyhydroxylated radiopaque compound into hydroxyaminoethyl groups (—CHOHCH₂NH₂ groups), or (iii) converting vicinal diol groups of a polyhydroxylated radiopaque compound into aminomethyl groups (—CH₂NH₂ groups), wherein the mixture is administered under conditions such that the amino groups of the radiopaque polyamino compound and the reactive moieties of the reactive polymer form covalent crosslinks after administration.
